(19) United States
(12) Patent Application Publication
(10) Pub. No.: US 2025/0110100 A1
(43) Pub. Date: Apr. 3, 2025

(54) SURFACE GEOCHEMICAL SURVEILLANCE OF TRACE ELEMENT ANOMALIES TO DELINEATE THE POTENTIAL OF PROSPECT OIL FIELDS

(71) Applicant: SAUDI ARABIAN OIL COMPANY, Dhahran (SA)

(72) Inventors: Peter BIRKLE, Dhahran (SA); Vyacheslav Viadislavovich GROKHOVSKY, Moscow (RU); Maxim Viadimirovich ORLOV, Moscow (SA); Waleed SAEED, Dhahran (SA)

(73) Assignee: SAUDI ARABIAN OIL COMPANY, Dhahran (SA)

(21) Appl. No.: 18/480,412

(22) Filed: Oct. 3, 2023

(51) Int. Cl. G01N 33/24; G06F 18/2321

(52) U.S. Cl. CPC G01N 33/24; G06F 18/2321

(57) ABSTRACT

A method for determining exploration potential of a prospect oil field includes determining microseepage at the prospect oil field by performing sequential extraction of trace elements (TE) and/or heavy metals (HM) from soil samples obtained at substantially surface level of the prospect oil field and dry and proven fields, visualizing results of the sequential extraction in isoline maps, using statistical clustering based on a machine learning model to detect concentration anomalies of the extracted trace elements (TE) and/or heavy metals (HM) in the isoline maps of the visualized results, correlating detected concentration anomalies among the prospect, dry and proven fields, and recommending for or against exploration of the prospect oil field depending on whether the concentration anomalies of the prospect field correlate with those of the proven field or the dry field.

### FIELD OF THE DISCLOSURE

The present disclosure relates generally to oil exploration, and, more particularly, to a tool to enhance hydrocarbon exploration and the presence of oil reserves through the detection of vertical migration and accumulation of inorganic trace elements in surface soil samples with a combination of geochemical, statistical and machine-learning methods.

### BACKGROUND OF THE DISCLOSURE

In conventional methods of petroleum exploration, indirect and less costly surface indicators can be utilized to trace vertical seepage of hydrocarbons and potential petroleum resources from the subsurface. Generally utilized surface surveillance methods are based on air and land sensing (satellite scanning, remote vehicles), computer-supported methods, geophysical tools (electrical anomalies, laser tools, radar waves) and organic geochemistry (clumped isotopes; gas and hydrocarbon analysis), which are briefly described as follows:

### Geophysics and Remote Sensing

Electrical anomalies: The mapping of electrical anomalies in the near surface reflect a shallow electrical expression of geochemical characteristics that may or not may be due to seepage from a deeper hydrocarbon reservoir (Oehler, 1982).

Electromagnetic waves: The comparison of reflected microwave signals with predetermined “microwave re-radiation characteristics” (MRC) of known gases allows the detection of escaping gases from underground stratigraphic and structural traps (Owen and Busby, 1972).

Laser methods: Besides its application for the detection of a wide range materials (i.e., vegetation, greenhouse gases, environmental pollutants, explosives, building materials), short-wave infrared (SWIR) laser can be utilized to detect natural gas leak with applications for exploration to detect methane and ethane (Islam, 2018).

Radar waves: Radar is used to detect and map near-surface geochemical alteration of rock and soil, whereby the radar waves penetrate a buildup of carbonate and silica on the earth surface above micro-seepage of hydrocarbons dissolved in water (Duren and Warren, 1995).

Satellite scanning: Infrared electromagnetic radiation signals from solar energy are recorded and filtered by satellites to detect hydrocarbon gas clouds above oil or gas reservoirs (Brame, 1989).

Remote vehicles: Bond and Pottorf (2017) describe the utilization of unmanned surface vehicles (USVs) to obtain samples for the identification of hydrocarbon systems.

### Organic Geochemistry

Geochemistry in submarine spills: Kennicutt et al. (1988) describes the analysis of organic isotopes in sea slicks and tar balls to trace natural gas seepage in a deepwater marine settings. Sassen et al. (2001) showed correlation of isotopic properties of C1-C5 hydrocarbon from reservoirs, gas vents, and gas hydrates in the Gulf of Mexico. Baksmaty et al. (2019) describe a methodology to determine physical, transport and thermodynamic fluid properties of hydrocarbon seeps at the seafloor.

Gas geochemistry: Romanak and Bennett (2015) present a methodology to utilize CO2, O2, CH4, and N2 levels from surface or near surface geological samples to determine the presence of natural in-situ CO2 or exogenous leakage input from deep reservoirs. Sterner et al. (2007) describe an apparatus to analyze samples of trap gas, mud fluid, or cuttings by mass spectrometer.

Biomarkers: Cold-shock genes (DNA, RNA, proteins or metabolites) from microbial organisms are identified in hydrocarbon seep to evaluate the presence of a hydrocarbon reservoir or a pipeline leakage (Pilloni and Summers, 2019).

Gas and hydrocarbon analysis: Hydrocarbon seepage can be detected by measuring reservoir hydrocarbon through soil-gas surveys. In practice, hydrocarbon sensors are positioned at different depths (1.0 m, 5.0 m, more than 5 m from surface) for gas sampling and subsequent organic analysis (Mahdi et al., 2016).

Gas sampling tool: Thompson (1981) describes the design of a gas probe to recover small subsurface gas samples from the soil for analysis during geochemical exploration. The probe shaft also includes temperature, soil humidity and soil pH sensors to obtain additional parameters.

Metal isotopes: Multicomponent metal isotope signatures (δ⁵⁴Cr, δ⁶⁵Cu, δ⁵⁸Fe, δ⁹⁸Mo, δ⁶⁰Ni, δ⁵¹V, δ⁶⁶Zn) are analyzed in source rock, drilling fluids or hydrocarbon fluids from subsurface or surface locations for different purposes, such as to trace the origin of hydrocarbons, migration pathways, and hydrocarbon-seep correlation (Formolo, 2018).

### Artificial Intelligence

Computer-supported methods: Predictive production maps of subsurface hydrocarbon sources and microseepage are created through correlation algorithm on microbial prospecting (Te Stroet et al., 2018).

Subsurface methods, such as reflection seismic, gravity or magnetics, are generally the dominant technology for the identification of trap structures and hydrocarbon accumulation. As a disadvantage, the application of geophysical exploration tools, especially 3D or 4D seismic, is very lengthy and costly (field equipment, field campaign, data processing). Other methods, such as well log interpretation or geochemical analysis of reservoir fluids are limited to an advanced stage in exploration, by having the requirement to utilize perforated drilling holes. Examples for the utilization of geochemical methods are the analysis of hydrocarbons in oil samples and pore space of rock samples (Liu and Wu, 2020), isotopic signatures from produced fluids, oil, gas and rock samples from the subsurface (Lawson et al., 2017), and clumped isotopes (Lawson et al., 2014).

### Inorganic Geochemical Surveillance Methods

Surficial geochemical exploration is a perspective approach for oil and gas surveying, which has been used since the 1930s (Schumacher, 2000). Geochemical surveillance completes classical seismic exploration and make surveying results more reliable and precise. For development projects, detailed microseepage surveys can (1) help evaluate infill or stepout drilling locations, (2) delineate productive limits of undeveloped fields, (3) identify bypassed pay or undrained reservoir compartments, and (4) monitor hydrocarbon drainage through use of repeat geochemical surveys.

The Mobile Metal Ion (MMI) technology is an analytical process that uses analysis of metals in soils and related materials. The technology was developed by SGS (www.sgsgroup.com.cn), whereby target elements are extracted using weak solutions of organic and inorganic compounds rather than conventional aggressive acid or cyanide-based digests. MMI solutions contain strong ligands, which detach and hold metal ions that were loosely bound to soil particles by weak atomic forces in aqueous solution. This extraction does not dissolve the bound forms of the metal ions. Metal ions in the MMI solutions are chemically active or ‘mobile’ component of the sample. Because these mobile, loosely bound complexes are in very low concentrations, measurement is conducted by conventional ICP-MS and the latest evolution of this technology, ICP-MS Dynamic Reaction Cell™ (DRC II™).

The Mobile Metal Ion (MMI®) analytical procedure is a surficial geochemical exploration tool that was used to assess its value in exploring for unconventional shallow shale gas reservoirs at a test site southwest of Manitou, Manitoba (Fedikow et al., 2009). The MMI technology was successfully applied there for hydrocarbon exploration along a transect (Fedikow et al., 2009, 2010). Historically, ligand soil-based extraction geochemistry was applied for the delineation of sub-surface mineralization, i.e., for Cu, Co and Zn in Butte Montana, USA, or the Green Tree Frog Prospect in Queensland, Australia (Mann, 2007). The Mobile Metal Ion technology has been limited to mining and gas exploration.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a method for determining exploration potential of a prospect oil field includes determining microseepage at the prospect oil field by performing sequential extraction of trace elements (TE) and/or heavy metals (HM) from soil samples obtained at substantially surface level of the prospect oil field and dry and proven fields, visualizing results of the sequential extraction in isoline maps, using statistical clustering based on a machine learning model to detect concentration anomalies of the extracted trace elements (TE) and/or heavy metals (HM) in the isoline maps of the visualized results, correlating detected concentration anomalies among the prospect, dry and proven fields, and recommending for or against exploration of the prospect oil field depending on whether the concentration anomalies of the prospect field correlate with those of the proven field or the dry field.

In another embodiment, a machine-readable storage medium having stored thereon a computer program for determining exploration potential of a prospect oil field from trace elements (TE) and/or heavy metals (HM) extracted from soil samples obtained at substantially surface level of the prospect oil field and dry and proven fields is disclosed. The computer program includes a routine of set instructions for causing the machine to perform the steps of visualizing results of the sequential extraction in isoline maps, using statistical clustering based on a machine learning model to detect concentration anomalies of the extracted trace elements (TE) and/or heavy metals (HM) in the isoline maps of the visualized results, correlating detected concentration anomalies among the prospect, dry and proven fields, recommending for or against exploration of the prospect oil field depending on whether the concentration anomalies of the prospect oil field correlate with those of the proven field or the dry field.

In a further embodiment, a system for determining exploration potential of a prospect oil field includes an interpreter for receiving extracted trace element (TE) and/or heavy metal (HM) data from soil samples obtained at substantially surface level of the prospect oil field and dry and proven fields. The interpreter has a visualizer including a 2D plotter for plotting received data in isoline maps, an analyzer having a clustering module and an anomaly detector that are operable to apply statistical clustering and machine-learning techniques, using a machine learning (ML) model, to group soil sample data and to detect concentration anomalies of specific elements therein, a correlator operable correlate concentration anomalies among the prospect, dry and proven fields, and a recommender operable to recommend for or against exploration of the prospect oil field depending on whether the concentration anomalies of the prospect field correlate with those of the proven field or the dry field.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow diagram of an exploration method for detection of vertical microseepage of crude oil in prospect oil fields.

FIG. 2 is a block diagram of a system 200 for determining oil field exploration potential in accordance with certain embodiments.

FIG. 3 is a map of soil sample distributions from proven fields (A-Field: 14 samples; H-Field: 10 samples; N-Field: 12 samples; R-Field: 32 samples), dry fields (K-Field: 10 samples; U-Field: 20 samples) and prospect field (J-Field: 10 samples) with a mass between 100 and 250 g.

FIG. 4 is a graph showing a comparison of AAB extraction values of Cu, Ni, Ti, V, and Co [μg/kg, ppb] for dry U-Field (UA1-UP3) and proven R-Field (RD1-RB13).

FIG. 5 shows concentration of Ni [μg/kg, ppb] from AAB extraction in prospect field (JA1-JE4=J-Field), dry fields (UA1-UP3=U-Field; KB1-KE4=K-Field), and proven fields (RA1-RB13=R-Field, NA1-NF3=N-Field; HA1-HE5=H-Field; AC1-AC7=A-Field).

FIG. 6 shows a comparison of highlighted elemental concentrations (As, Cu, Ni, Ti, V in μg/kg or ppb) from AAEB soil extraction between the dry K-Field (KB1-KE3) and proven R-Field (RD3-RC8).

FIG. 7 is an isoline map with concentration of arsenic in surface soil samples from proven, dry and prospect fields in the studied exploration area.

FIG. 8 is a comparison of arsenic concentrations in surface soil samples from proven, dry and prospect fields in a specific exploration area.

FIG. 9 is an isoline map with concentration of titanium in surface soil samples from proven, dry and prospect fields in a specific exploration area.

FIG. 10 is a comparison of titanium concentrations in surface soil samples from proven, dry and prospect fields in a specific exploration area.

FIG. 11 is a block diagram of a computer system that may be used to implement one or more of the systems or methods described herein in accordance with certain embodiments.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to oil exploration, and, more particularly, to a tool to enhance hydrocarbon exploration and the presence of oil reserves through the detection of vertical migration and accumulation of inorganic trace elements in surface soil samples with a combination of geochemical, statistical and machine-learning methods. Embodiments disclosed herein differ significantly from conventional MMI technology, for example deploying in a novel manner geochemical techniques in combination with exploration assessments of prospect fields by statistical and machine-learning methods to detect microseepage in oil fields. Novel geochemical techniques include soil sampling in prospect, proven and dry fields, sampling of reference oil, novel soil extraction techniques, and analysis of trace elements and heavy metals.

In accordance certain embodiments, an inorganic geochemical surveillance technique can be applied to detect vertical microseepage from deep oil resources. The technology can be utilized as a prediction tool to delineate the potential of exploration areas prior to drilling. In contrast to MMI, the techniques described herein utilize a multi-disciplinary approach of soil sampling, sequential extraction techniques, geochemical analysis of trace elements and metals, and analyzed concentrations of trace elements (TE) and heavy metals (HM), which are plotted as 2D visualization maps and histograms, and to which statistical clustering may be applied. Machine-learning techniques are applied to group soil samples and to detect concentration anomalies of specific elements. A final assessment of geochemical properties from prospect, proven and dry fields to quantify the probability for the detection of oil resources in proven fields is made.

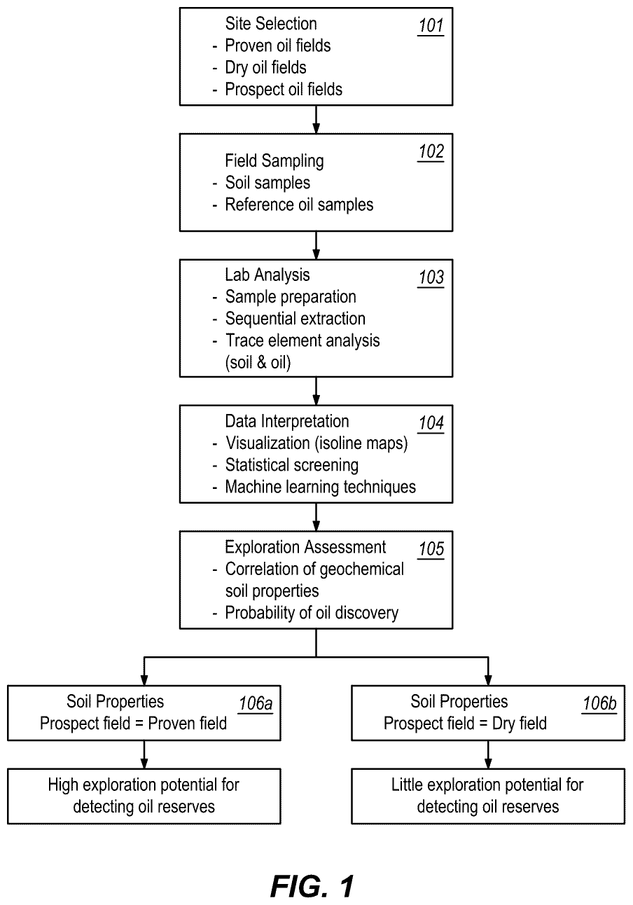

FIG. 1 is a flow diagram of an exploration method 100 for detection of vertical microseepage of crude oil in prospect oil fields. Generally, using method 100, the concentration of trace elements in surface soil samples in prospect fields is correlated with compositional features in proven and dry fields. At 101, site selection is performed. Selected sites are from one or more of a proven oil field, dry oil field, and the prospect oil field under consideration. As an example, a delineation of a sample grid for a prospective oil field, and a series of adjacent proven and dry fields can be made.

At 102, field sampling is conducted and reference soil samples are obtained. Field sampling can entail obtaining soil samples from the proven, dry and prospect oil fields. In one example, a sampling grid (i.e., 100 m×100 m) is designed for each field. At each site, a sample mass between about 150 and 450 g is taken from a depth of about 10-40 cm, using a manual drilling instrument for instance, and stored in sealed plastic bags. As reference samples for the abundance of trace elements in crude oil, oil samples can be collected from exploration or development wells in proven fields.

Lab analysis is conducted at 103. This includes sample preparation, sequential extraction and trace element analysis of soil, plus sample preparation and trace element analysis of oil. Generally, soil samples are enriched as solid phase in major elements (Si, Fe, C, Ca) due to their high content in the soil. In contrast, trace elements (TE) and heavy metals (HM) are found in soil mainly as impurities in various mineral and organic soil components. Accordingly, it is not always possible to have predetermined conditions for the selective extraction of individual TE and HM compounds from the soil. Therefore, the TE and HM content is commonly determined by dissolving the soil in an extracting solution to be separated into fractions according to the strength of the bond with solid-phase soil components. In one example, the following series of extraction processes is performed for the soil samples. The extraction of trace elements is performed with the extraction solutions indicated:
Stage 1: Deionized water for water-soluble forms of TE and HM
Stage 2: Nitric acid (HNO₃) for acid-soluble forms of TE and HM
Stage 3: Acetate-ammonium buffer (AAB)
Stage 4: Mixed solution with weak organic acids

At 104, interpretation of the geochemical data is conducted. Visualization of the results, for example in isoline maps, as well as statistical screening and machine learning techniques are applied in this step. In certain embodiments, analyzed concentrations of TE and HM are plotted as 2D visualization maps and histograms. Statistical clustering and machine-learning techniques can be applied to group soil samples and to detect concentration anomalies of specific elements.

At 105, an exploration assessment is performed. This can include correlation of geochemical soil properties to determine a probability of oil discovery, whereby at 106a, if the properties of the prospect field properly correlate to the those of the proven field, a high exploration potential is recommended; whereas, at 106b, if the properties of the prospect field properly correlate to the those of the dry field, a low exploration potential is recommendation. Specifically, geochemical properties of prospect fields are correlated with proven and dry fields to assess the probability for the detection of oil resources in proven fields. The data is normalized and a response index is calculated to estimate the values of the content of elements in various fields. The index is calculated as the ratio of an elemental concentration in a perspective field to its background concentration in a dry field. In case of geochemical affinities between the prospect fields and proven fields, an elevated exploration potential is given for the existence of oil reserves (106a). In contrast, geochemical similarities between soil samples from prospect fields and dry fields suggest little exploration potential for the studied prospect field (106b).

While, for purposes of simplicity of explanation, the example method of FIG. 1 is shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

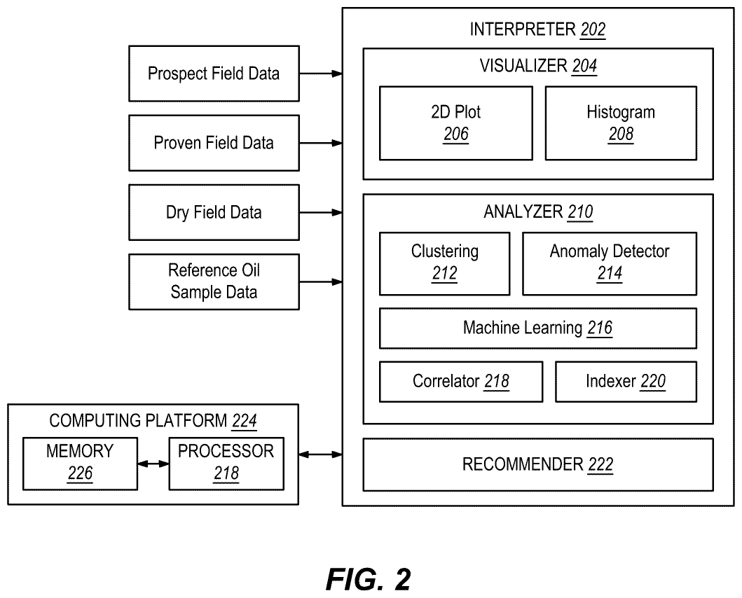

FIG. 2 is a block diagram of a system 200 for determining oil field exploration potential in accordance with certain embodiments. System 200 receives as inputs data from a prospect field, a proven field, and a dry field, as well as reference oil sample data. The data relates to trace elements and heavy metals as collected and extracted in the manner described above.

System 200 includes an interpreter 202 having a visualizer 204 that is operable to plot the received data in two dimensions (2D), for example as isoline maps using a 2D plotter 206; and as a histogram, using histogram generator 208, respectively.

Interpreter 202 also includes an analyzer 210 having a clustering module 212 and an anomaly detector 214 that are operable to apply statistical clustering and machine-learning techniques, using a machine learning (ML) model 216, to group soil sample data and to detect concentration anomalies of specific elements therein. A correlator 218 is operable to correlate geochemical properties of prospect fields with proven and dry fields to assess the probability for the detection of oil resources in proven fields. The results of the correlator 216 are provided to an indexer 218 which normalizes the data and calculates a response index as the ratio of an elemental concentration in a perspective field to its background concentration in a dry field.

Interpreter 202 further includes a recommender 222, which provides a recommendation output of the interpreter. Specifically, in the case of sufficient geochemical affinities between the prospect fields and proven fields, an elevated exploration potential is given for the existence of oil reserves by recommender 222. In contrast, geochemical similarities between soil samples from prospect fields and dry fields suggest little exploration potential for the studied prospect field, and a recommender indication to that effect is issued.

It will be appreciated that one or more of the interpreter components, such as the visualizer 204, analyzer 210, and the recommender 222, can be implemented (e.g., as machine readable instructions) on a computing platform 224. The computing platform 224 can include one or more computing devices selected from, for example, a desktop computer, a server, a controller, a blade, a mobile phone, a tablet, a laptop, a personal digital assistant (PDA), and the like. The computing platform 224 can include a memory 226 and a processor 228. By way of example, the memory 226 can be implemented, for example, as a non-transitory computer storage medium, such as volatile memory (e.g., random access memory), non-volatile memory (e.g., a hard disk drive, a solid-state drive, a flash memory, or the like), or a combination thereof. The processor 228 can be implemented, for example, as one or more processor cores.

The memory 226 can store machine-readable instructions that can be retrieved and executed by the processor 228. Each of the processor 228 and the memory 226 can be implemented on a similar or a different computing platform. The computing platform 224 can be implemented in a cloud computing environment (for example, as disclosed herein) and thus on a cloud infrastructure. In such a situation, features of the computing platform 224 can be representative of a single instance of hardware or multiple instances of hardware executing across the multiple of instances (e.g., distributed) of hardware (e.g., computers, routers, memory, processors, or a combination thereof). Alternatively, the computing platform 224 can be implemented on a single dedicated server or workstation.

### EXAMPLE

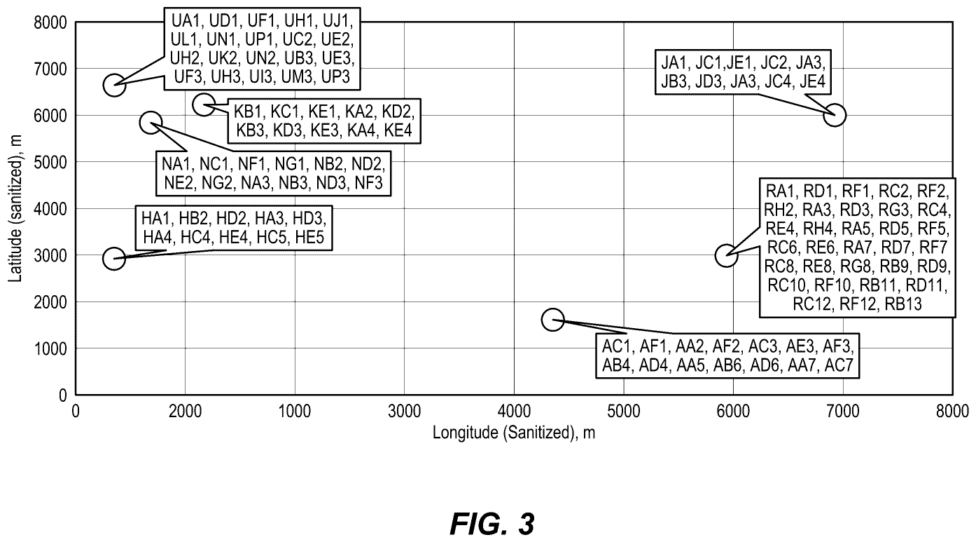

1. Field sampling campaign: During a field campaign, 180 soil samples were recovered from five proven oil fields (A-, D-, H-, N-, R-Field), 160 soil samples from three dry fields (K-, M-, U-Field), and 20 soil samples from one prospect field (J-Field). The distance between the sampling sites within each field was in the range of 10-20 m. At each site, a sample volume between 150 and 450 g was taken from a depth of 10-40 cm, using a drilling instrument (not shown). Out of the total sample batch, 108 soil samples (FIG. 3) from proven fields (A-Field: 14 samples; H-Field: 10 samples; N-Field: 12 samples; R-Field: 32 samples), dry fields (K-Field: 10 samples; U-Field: 20 samples) and prospect field (J-Field: 10 samples) with a mass between 100 and 250 g were selected for the subsequent sequential extraction and analytical process.

### 2. Sequential Extraction and Analysis

a) Soil sample preparation and hygroscopic moisture evaluation.
b) Preparing extracting agent solutions.
c) Obtaining extracts from soil samples.
   The extraction of trace elements was performed with following extraction solutions:
   Deionized water
   Nitric acid (HNO₃)
   Acetate-ammonium buffer (AAB)
   Mixed solution with weak organic acids
d) Calibrating the ICP-OES instrument using standards.
e) Measuring the extracts and receiving the final data set with concentrations of trace elements.

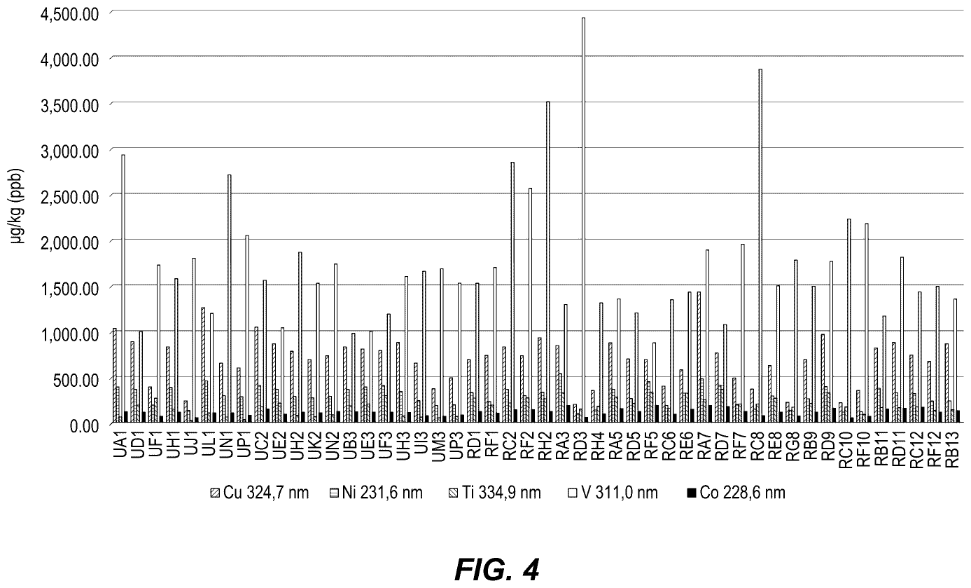

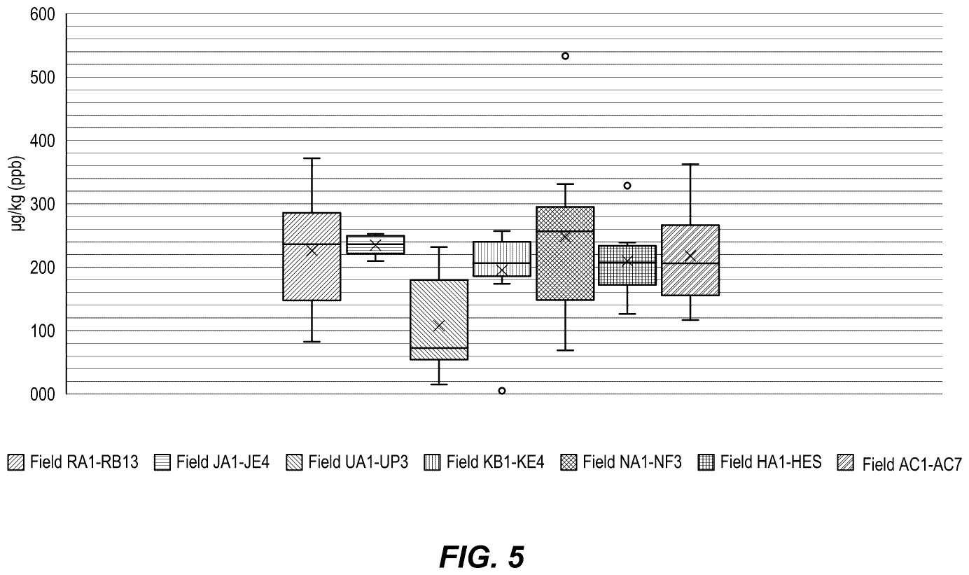

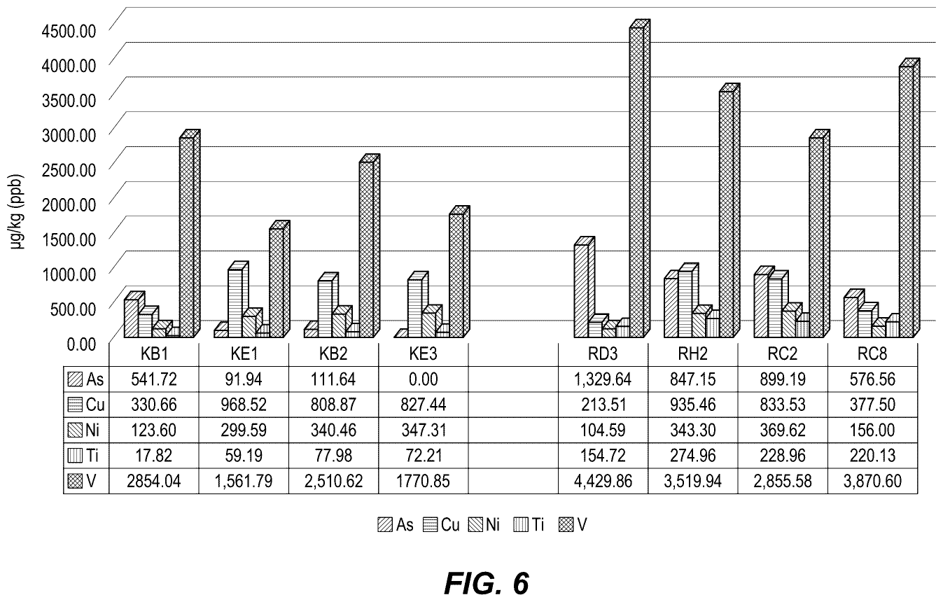

3. Geochemical Assessment: The extraction by using ammonium-acetate buffer solution showed a higher resolution compared to aqueous and nitric acid extractions. This is achieved due to the selective effect on the soil substrate. Comparing the soil composition of the dry U-Field (sample sites UA1-UP3) with the proven R-Field (RA1-RB13), elements such as Ni, V, Ti, and Cr show the greatest variability in content. FIG. 4 demonstrates that soil samples from R-Field (RD1-RB13) contain outstanding values for several elements. Ni concentrations [μg/kg, ppb] from AAB extraction in the prospect J-Field are generally elevated in comparison to dry fields (UA1-UP3=U-Field; KB1-KE4=K-Field), but within the range of proven fields (RA1-RB13=R-Field, NA1-NF3=N-Field; HA1-HE5=H-Field, AC1-AC7=A-Field) (FIG. 5). As an example, the vanadium content at the locations RD3, RH2, RC2, RC8 is 2-2.5 times higher than values of other field locations and contrasts with the points KB1, KE1, KD2 KE3 from the dry K-Field (FIG. 6).

### 4. Spatial Visualization and Hot Spot Detection

Anomalies in the elemental concentrations, extracted with a mixed solution of weak organic acids (stage 4), were utilized for the characterization of potential microseepage processes and compositional distinction between proven, dry and prospect fields. As main outcomes, specially the elements V, Ti, and As resulted to be most differentiated within the wide range of analyzed trace elements (Al, As, Cd, Co, Cr, Cu, Eu, Fe, La, Mn, Mo, Ni, P, Pb, Sb, Sn, Tb, Ti, V, Yb, Zn).

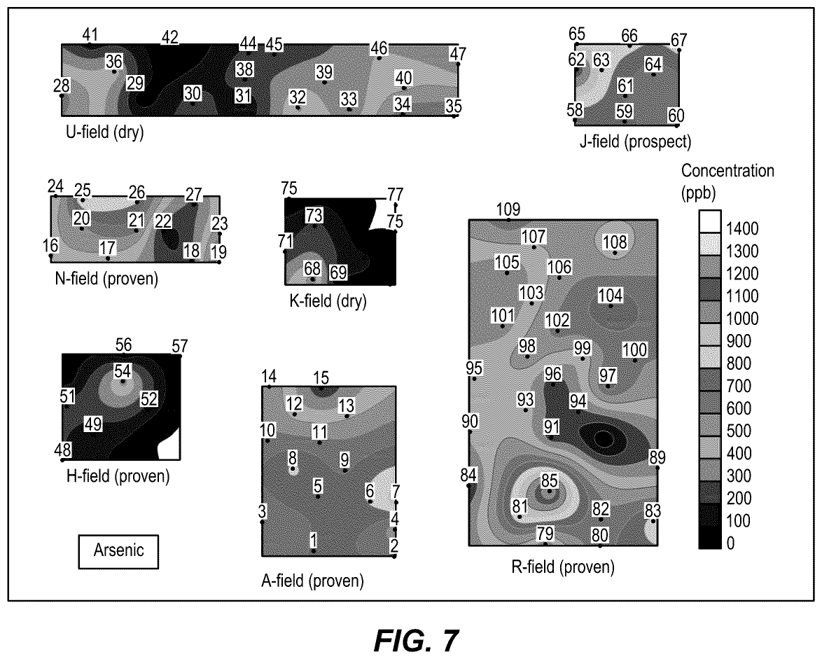

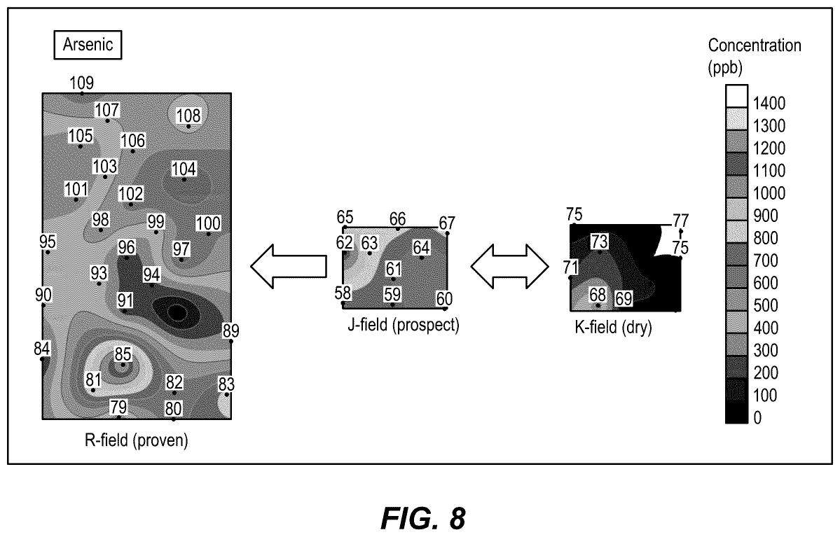

The isoline maps in FIGS. 7 to 10 illustrate the compositional variation of As and Ti concentrations for analyzed soil samples in the analyzed fields. In case of arsenic, the proven R-, A- and N-Fields show relatively homogeneous and elevated peak concentrations of 1,329 ppbₘₐₓ (red color), 959 ppbₘₐₓ, and 914 ppbₘₐₓ (orange color) respectively, while the proven H-Field (545 ppbₘₐₓ) is within the depleted range of the dry U-(704 ppb) and K-fields (541 ppbₘₐₓ) (FIG. 7). The prospect J-Field has one singe sampling point (JA3) with an elevated arsenic concentration of 1,188 ppbₘₐₓ, similarly to maximum peaks at proven fields. The arsenic concentration range of soil samples from J-Field (604-1,188 ppb) is most similar to the proven R-Field (215-1,329 ppb) and A-Field (215-959 ppb), which could reflect vertical microseepage at the prospect J-Field. The dry U-Field (0-704 ppb) and K-Field (0-541 ppb) are mostly depleted in arsenic. As shown in FIG. 8, the intermediate (green color) to elevated (red color) arsenic concentrations of the prospect J-Field are closer related to concentration peaks in the proven R-Field as to depleted values (blue color) in the dry K-Field.

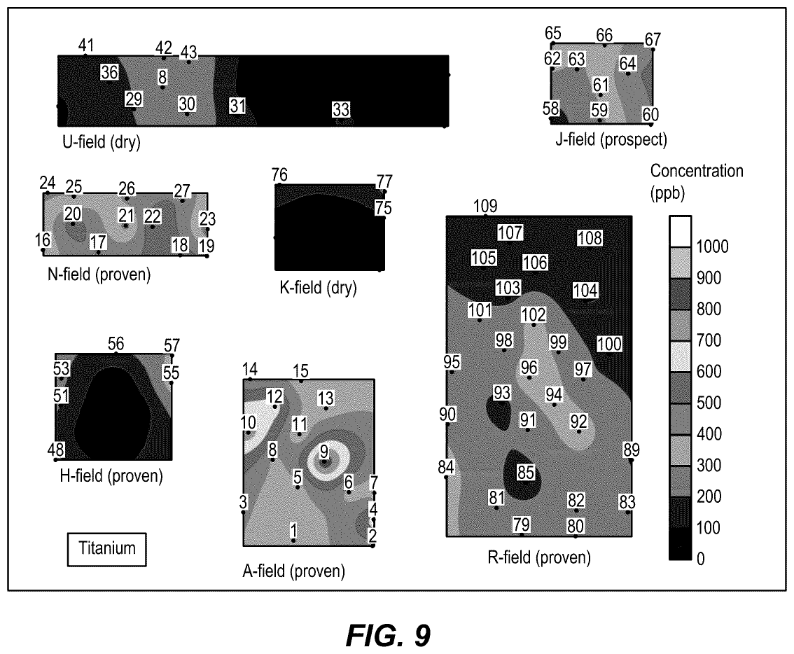

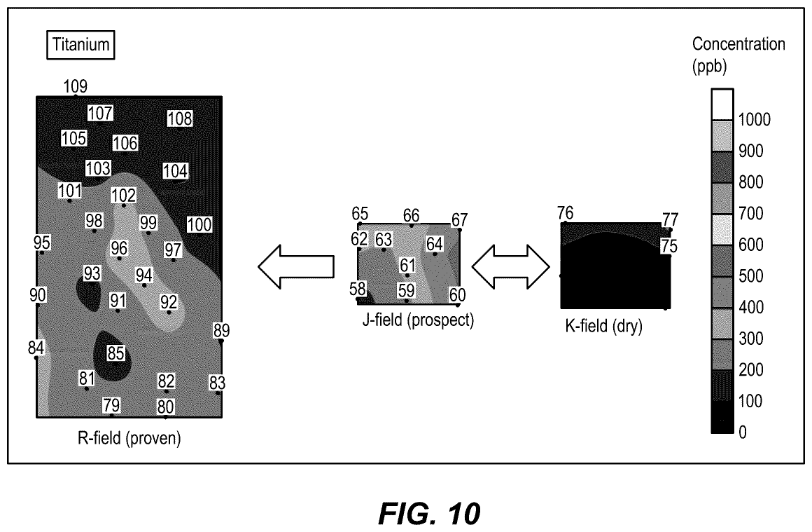

Maximum titanium concentrations are diverse in proven fields, from highest peaks at A-(919 ppbₘₐₓ) and N-(589 ppbₘₐₓ) to lowest at R-(381 ppbₘₐₓ) and H-Field (367 ppbₘₐₓ) (FIG. 9). In contrast, the dry U-(307 ppbₘₐₓ) and K-Field (220 ppbₘₐₓ) are generally depleted in titanium, while the prospect J-Field (503 ppbₘₐₓ) falls within the range of proven fields (FIG. 10). In contrast to the dry fields with a concentration range from 28 ppb to 307 ppb for U-Field and 0 ppb to 220 ppb for K-Field, the prospect J-Field show a general presence of titanium with a value range from 159 ppb to 503 ppb. It is therefore likely, that the accumulation of titanium at J-Field has a similar provenance as in A-, N- and R-proven fields.

Microseepage assessment: It can be concluded that especially compositional differences between As- and Ti-depleted soils in dry fields and As- and Ti-enriched soils in proven fields suggest that J-Field is more prone to proven fields with the potential presence of microseepage in latter fields. The heterogenous abundance of specific elements, even within a proven field, suggest that vertical migration and deposit of trace elements does occur on a local scale, likely aligned to structural features.

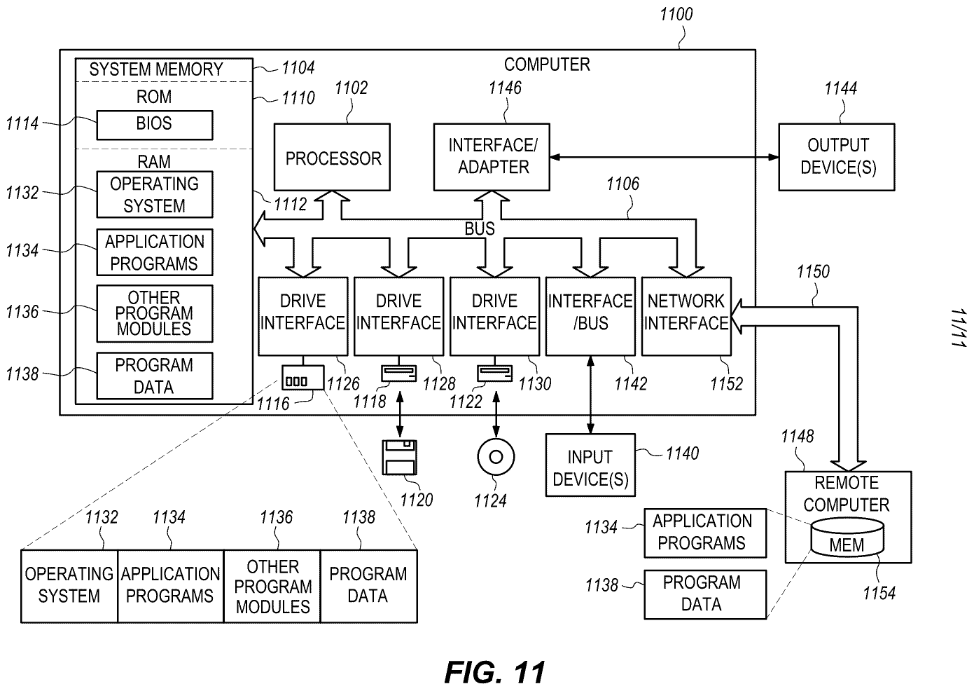

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 11. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 11 illustrates one example of a computer system 1100 that can be employed to execute one or more embodiments of the present disclosure. Computer system 1100 can be implemented on one or more general purpose networked computer systems, embedded computer systems, routers, switches, server devices, client devices, various intermediate devices/nodes or standalone computer systems. Additionally, computer system 1100 can be implemented on various mobile clients such as, for example, a personal digital assistant (PDA), laptop computer, pager, and the like, provided it includes sufficient processing capabilities.

Computer system 1100 includes processing unit 1102, system memory 1104, and system bus 1106 that couples various system components, including the system memory 1104, to processing unit 1102. System memory 1104 can include volatile (e.g. RAM, DRAM, SDRAM, Double Data Rate (DDR) RAM, etc.) and non-volatile (e.g. Flash, NAND, etc.) memory. Dual microprocessors and other multi-processor architectures also can be used as processing unit 1102. System bus 1106 may be any of several types of bus structure including a memory bus or memory controller, a peripheral bus, and a local bus using any of a variety of bus architectures. System memory 1104 includes read only memory (ROM) 1110 and random access memory (RAM) 1112. A basic input/output system (BIOS) 1114 can reside in ROM 1110 containing the basic routines that help to transfer information among elements within computer system 1100.

Computer system 1100 can include a hard disk drive 1116, magnetic disk drive 1118, e.g., to read from or write to removable disk 1120, and an optical disk drive 1122, e.g., for reading CD-ROM disk 1124 or to read from or write to other optical media. Hard disk drive 1116, magnetic disk drive 1118, and optical disk drive 1122 are connected to system bus 1106 by a hard disk drive interface 1126, a magnetic disk drive interface 1128, and an optical drive interface 1130, respectively. The drives and associated computer-readable media provide nonvolatile storage of data, data structures, and computer-executable instructions for computer system 1100. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 1110, including operating system 1132, one or more application programs 1134, other program modules 1136, and program data 1138. In some examples, the application programs 1134 can include visualizer 204, analyzer 210 and recommender 222, and the program data 1138 can include the results of the sequential extraction which can include prospect field data, proven field data, dry field data, and reference oil sample data. The application programs 1134 and program data 1138 can include functions and methods programmed to determine exploration potential of a prospect oil field and to determine microseepage at the prospect oil field, such as shown and described herein.

A user may enter commands and information into computer system 1100 through one or more input devices 1140, such as a pointing device (e.g., a mouse, touch screen), keyboard, microphone, joystick, game pad, scanner, and the like. These and other input devices 1140 are often connected to processing unit 1102 through a corresponding port interface 1142 that is coupled to the system bus, but may be connected by other interfaces, such as a parallel port, serial port, or universal serial bus (USB). One or more output devices 1144 (e.g., display, a monitor, printer, projector, or other type of displaying device) is also connected to system bus 1106 via interface 1146, such as a video adapter.

Computer system 1100 may operate in a networked environment using logical connections to one or more remote computers, such as remote computer 1148. Remote computer 1148 may be a workstation, computer system, router, peer device, or other common network node, and typically includes many or all the elements described relative to computer system 1100. The logical connections, schematically indicated at 1150, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 1100 can be connected to the local network through a network interface or adapter 1152. When used in a WAN networking environment, computer system 1100 can include a modem, or can be connected to a communications server on the LAN. The modem, which may be internal or external, can be connected to system bus 1106 via an appropriate port interface. In a networked environment, application programs 1134 or program data 1138 depicted relative to computer system 1100, or portions thereof, may be stored in a remote memory storage device 1154.

Embodiments disclosed herein include:

A. A method for determining exploration potential of a prospect oil field, the method comprising:
determining microseepage at the prospect oil field by performing sequential extraction of trace elements (TE) and/or heavy metals (HM) from soil samples obtained at substantially surface level of the prospect oil field and dry and proven fields;
visualizing results of the sequential extraction in isoline maps;
using statistical clustering based on a machine learning model to detect concentration anomalies of the extracted trace elements (TE) and/or heavy metals (HM) in the isoline maps of the visualized results;
correlating detected concentration anomalies among the prospect, dry and proven fields; and
recommending for or against exploration of the prospect oil field depending on whether the concentration anomalies of the prospect field correlate with those of the proven field or the dry field.

B. A machine-readable storage medium having stored thereon a computer program for determining exploration potential of a prospect oil field from trace elements (TE) and/or heavy metals (HM) extracted from soil samples obtained at substantially surface level of the prospect oil field and dry and proven fields, the computer program comprising a routine of set instructions for causing the machine to perform the steps of:
visualizing results of the sequential extraction in isoline maps;
using statistical clustering based on a machine learning model to detect concentration anomalies of the extracted trace elements (TE) and/or heavy metals (HM) in the isoline maps of the visualized results;
correlating detected concentration anomalies among the prospect, dry and proven fields; and
recommending for or against exploration of the prospect oil field depending on whether the concentration anomalies of the prospect oil field correlate with those of the proven field or the dry field.

C. A system for determining exploration potential of a prospect oil field, the system comprising:
an interpreter for receiving extracted trace element (TE) and/or heavy metal (HM) data from soil samples obtained at substantially surface level of the prospect oil field and dry and proven fields, the interpreter including:
   a visualizer including a 2D plotter for plotting received data in isoline maps;
   an analyzer having a clustering module and an anomaly detector that are operable to apply statistical clustering and machine-learning techniques, using a machine learning (ML) model, to group soil sample data and to detect concentration anomalies of specific elements therein;
   a correlator operable correlate concentration anomalies among the prospect, dry and proven fields; and
   a recommender operable to recommend for or against exploration of the prospect oil field depending on whether the concentration anomalies of the prospect field correlate with those of the proven field or the dry field.

Each of embodiments A through C may have one or more of the following additional elements in any combination: Element 1: wherein the extraction is performed in multiple stages as follows:
Stage 1: Deionized water for water-soluble forms of TE and HM,
Stage 2: Nitric acid (HNO₃) for acid-soluble forms of TE and HM,
Stage 3: Acetate-ammonium buffer (AAB),
Stage 4: Mixed solution with weak organic acids.

Element 2: wherein visualizing is further conducted using histograms.

Element 3: wherein the soil samples are obtained from a depth of about 10-40 cm.

Element 4: wherein the soil samples have a mass of about 150-450 grams.

Element 5: wherein the trace elements (TE) and/or heavy metals (HM) include As and Ti.

Element 6: wherein the trace elements (TE) and/or heavy metals (HM) include one or more of As, Ti, Co, Cu, Ni, and V.

By way of non-limiting example, exemplary combinations applicable to A through C include: Element 1 with Element 2; Element 2 with Element 3; Element 3 with Element 4; Element 2 with Element 5; Element 1 with Element 5; Element 2 with Element 4; and Element 4 with Element 5.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by thosue skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A method for determining exploration potential of a prospect oil field, the method comprising:
determining microseepage at the prospect oil field by performing sequential extraction of trace elements (TE) and/or heavy metals (HM) from soil samples obtained at substantially surface level of the prospect oil field and dry and proven fields;
visualizing results of the sequential extraction in isoline maps;
using statistical clustering based on a machine learning model to detect concentration anomalies of the extracted trace elements (TE) and/or heavy metals (HM) in the isoline maps of the visualized results;
correlating detected concentration anomalies among the prospect, dry and proven fields; and
recommending for or against exploration of the prospect oil field depending on whether the concentration anomalies of the prospect field correlate with those of the proven field or the dry field.

2. The method of claim 1, wherein the extraction is performed in multiple stages as follows:
Stage 1: Deionized water for water-soluble forms of TE and HM,
Stage 2: Nitric acid (HNO₃) for acid-soluble forms of TE and HM,
Stage 3: Acetate-ammonium buffer (AAB),
Stage 4: Mixed solution with weak organic acids.

3. The method of claim 1, wherein visualizing is further conducted using histograms.

4. The method of claim 1, wherein the soil samples are obtained from a depth of about 10-40 cm.

5. The method of claim 1, wherein the soil samples have a mass of about 150-450 grams.

6. The method of claim 1, wherein the trace elements (TE) and/or heavy metals (HM) include As and Ti.

7. The method of claim 1, wherein the trace elements (TE) and/or heavy metals (HM) include one or more of As, Ti, Co, Cu, Ni, and V.

8. A machine-readable storage medium having stored thereon a computer program for determining exploration potential of a prospect oil field from trace elements (TE) and/or heavy metals (HM) extracted from soil samples obtained at substantially surface level of the prospect oil field and dry and proven fields, the computer program comprising a routine of set instructions for causing the machine to perform the steps of:
visualizing results of the sequential extraction in isoline maps;
using statistical clustering based on a machine learning model to detect concentration anomalies of the extracted trace elements (TE) and/or heavy metals (HM) in the isoline maps of the visualized results;
correlating detected concentration anomalies among the prospect, dry and proven fields; and
recommending for or against exploration of the prospect oil field depending on whether the concentration anomalies of the prospect oil field correlate with those of the proven field or the dry field.

9. The machine-readable storage medium of claim 8, wherein the extraction is performed in multiple stages as follows:
Stage 1: Deionized water for water-soluble forms of TE and HM,
Stage 2: Nitric acid (HNO₃) for acid-soluble forms of TE and HM,
Stage 3: Acetate-ammonium buffer (AAB),
Stage 4: Mixed solution with weak organic acids.

10. The machine-readable storage medium of claim 8, wherein visualizing is further conducted using histograms.

11. The machine-readable storage medium of claim 8, wherein the soil samples are obtained from a depth of about 10-40 cm.

12. The machine-readable storage medium of claim 8, wherein the soil samples have a mass of about 150-450 grams.

13. The machine-readable storage medium of claim 8, wherein the trace elements (TE) and/or heavy metals (HM) include As and Ti.

14. The machine-readable storage medium of claim 8, wherein the trace elements (TE) and/or heavy metals (HM) include one or more of As, Ti, Co, Cu, Ni, and V.

15. A system for determining exploration potential of a prospect oil field, the system comprising:
an interpreter for receiving extracted trace element (TE) and/or heavy metal (HM) data from soil samples obtained at substantially surface level of the prospect oil field and dry and proven fields, the interpreter including:
a visualizer including a 2D plotter for plotting received data in isoline maps;
an analyzer having a clustering module and an anomaly detector that are operable to apply statistical clustering and machine-learning techniques, using a machine learning (ML) model, to group soil sample data and to detect concentration anomalies of specific elements therein;
a correlator operable correlate concentration anomalies among the prospect, dry and proven fields; and
a recommender operable to recommend for or against exploration of the prospect oil field depending on whether the concentration anomalies of the prospect field correlate with those of the proven field or the dry field.

16. The system of claim 15, wherein the visualizer further includes a histogram plotter for generating histograms from the received data.

17. The system of claim 15, wherein the soil samples are obtained from a depth of about 10-40 cm.

18. The system of claim 15, wherein the soil samples have a mass of about 150-450 grams.

19. The system of claim 15, wherein the trace elements (TE) and/or heavy metals (HM) include As and Ti.

20. The system of claim 15, wherein the trace elements (TE) and/or heavy metals (HM) include one or more of As, Ti, Co, Cu, Ni, and V.
